(19) United States
(12) Patent Application Publication
(10) Pub. No.: US 2023/0363984 A1
(43) Pub. Date: Nov. 16, 2023

(54) DENTAL COMPOSITION

(71) Applicant: TERAUCHI ENDO institute, Tokyo (JP)

(72) Inventor: Yoshitsugu TERAUCHI, Tokyo (JP)

(73) Assignee: TERAUCHI ENDO institute, Tokyo (JP)

(21) Appl. No.: 18/246,494

(22) PCT Filed: Sep. 24, 2021
(86) PCT No.: PCT/JP2021/035164

§ 371 Date: Mar. 23, 2023

(30) Foreign Application Priority Data
Sep. 23, 2020 (WO) PCT/JP2020/035734

(51) Int. Cl. A61K 6/851; A61K 6/17; A61K 6/54; A61K 6/70

(52) U.S. Cl. CPC A61K 6/851; A61K 6/17; A61K 6/54; A61K 6/70

(57) ABSTRACT

The present invention is a dental composition comprising a cement component and at least one selected from the group consisting of titanium oxide, pure titanium, sodium titanate, and strontium titanate.

### TECHNICAL FIELD

The present invention relates to a dental composition.

### BACKGROUND ART

In recent years, dental Portland cement referred to as MTA (Mineral Trioxide Aggregate) has come onto the market as a dental composition that is said to have hard tissue inducibility, and it is beginning to be used for disease of dental pulp or periapical tissue.

MTA is prepared by micropulverizing, for dental use, Portland cement that is being used for concrete or the like and adding an inorganic substance having radiopacity so that it can be used in the oral cavity, and it is hardened by mixing it with water through hydration reaction. This hardened body has excellent biocompatibility and hard tissue inducibility, and it has been also reported that the hardened body can form new hard tissue in dental pulp or root periodontal tissue, and the hardened body is used as a substitute for a calcium hydroxide pharmaceutical that is a pulp capping material or is used for various clinical applications, such as retrograde filling of root canal, perforation repair and apexification. As general MTA, MTA containing bismuth oxide as an X-ray contrast medium is known (Patent Literature 1), but it has a problem that it turns black when bismuth oxide is exposed to light.

In recent years, MTA to which zirconium oxide having been used in living bodies was added as an X-ray contrast medium, such as a dental material or an artificial joint, has been reported (Non Patent Literature 1). Zirconium oxide is a material that is not easily discolored because it is chemically stable, and MTA containing this is also said to be a material that is not easily discolored.

### CITATION LIST

### Patent Literature

Patent Literature 1
Japanese Patent Laid-Open No. 2019-127452

### Non Patent Literature

Non Patent Literature 1
Hungaro Duarte, M. A., de Oliveira El Kadre, G. D., Vivan, R. R., et al. (2009) Radiopacity of portland cement associated with different radiopacifying agents. Journal of Endodontics 35(5), 737-40. doi: 10.1016/j.joen.2009.02.006

### SUMMARY OF INVENTION

### Technical Problem

It has been reported that use of zirconium oxide as an X-ray contrast medium exerts an effect of increasing compression strength or shortening the setting time as compared with a case of using bismuth oxide. However, when zirconium oxide is used as an X-ray contrast medium, there is a problem of insufficient contrast properties.

In the light of the above circumstances, it is an object of the present invention to provide a dental composition that is excellent in balance among all of contrast properties, discoloration resistance and compression strength and is substitutable for the conventional MTA.

In order to solve the above problem, the present inventors have earnestly studied. As a result, they have found that the above problem can be solved by using a composition comprising a cement component and at least one selected from the group consisting of titanium oxide, pure titanium, sodium titanate, and strontium titanate, and they have completed the present invention.

That is to say, the present invention is as follows.

[1]

A dental composition comprising
a cement component, and
at least one selected from the group consisting of titanium oxide, pure titanium, sodium titanate, and strontium titanate.
   [2]

The dental composition according to the above [1], wherein the cement component is Portland cement.

[3]

The dental composition according to the above [1] or [2], wherein an average particle diameter of the cement component is 1 to 100,000 nm.

[4]

The dental composition according to any one of the above [1] to [3], wherein the cement component contains substantially no tetracalcium aluminoferrite.

[5]

The dental composition according to any one of the above [1] to [4], wherein a content of the cement component is 50 to 95 mass %, and a content of the at least one selected from the group consisting of titanium oxide, pure titanium, sodium titanate, and strontium titanate is 5 to 30 mass %, based on the total solids of the dental composition.

[6]

The dental composition according to any one of the above [1] to [5], wherein a content of the at least one selected from the group consisting of titanium oxide, pure titanium, sodium titanate, and strontium titanate is 10 to 25 mass %.

[7]

The dental composition according to any one of the above [1] to [6], wherein an average particle diameter of the titanium oxide is 3 to 100 nm.

[8]

The dental composition according to any one of the above [1] to [7], wherein a content of titanium in the pure titanium is 99.9 mass % or more.

[9]

The dental composition according to any one of the above [1] to [8], wherein an average particle diameter of the pure titanium is 1 nm to 300 μm.

[10]

The dental composition according to any one of the above [1] to [9], further comprising at least one calcium sulfate-containing inorganic substance selected from the group consisting of calcium sulfate dihydrate, calcium sulfate hemihydrate, anhydrous calcium sulfate, and gypsum.

[11]

The dental composition according to the above [10], wherein a content of the calcium sulfate-containing inorganic substance based on the total solids of the dental composition is 0 to 10 mass %.

[12]

The dental composition according to the above [10], wherein a content of the calcium sulfate-containing inorganic substance based on the total solids of the dental composition is 2 to 8 mass %.

[13]

The dental composition according to any one of the above [1] to [12], used as a root canal treatment material.

### Advantageous Effect of Invention

The dental composition of the present invention exerts an effect of having all of excellent contrast properties, discoloration resistance and compression strength.

### DESCRIPTION OF EMBODIMENT

Hereinafter, an embodiment for carrying out the present invention (abbreviated to “present embodiment” hereinafter) will be described in detail. However, the present invention is not limited to the following embodiment, and various modifications can be made within the scope of the present invention.

[Dental Composition]

The dental composition of the present embodiment comprises a cement component and at least one selected from the group consisting of titanium oxide, pure titanium, sodium titanate, and strontium titanate.

(Cement Component)

The cement component is not particularly restricted as long as it has biocompatibility, but from the viewpoints of adhesion properties and strength, it is preferably Portland cement, and it may be a substance obtained by appropriately refining Portland cement so that it can be used for dental applications, the Portland cement being generally used for civil engineering and construction applications.

The Portland cement is mainly composed of tricalcium silicate (alite, 3CaO·SiO₂), dicalcium silicate (belite, 2CaO·SiO₂), calcium aluminate (aluminate, 3CaO·Al₂O₃), and calcium aluminoferrite (ferrite, 4CaO·Al₂O₃·Fe₂O₃). The major components of these are calcium oxide (CaO), silicon dioxide (SiO₂), aluminum oxide (Al₂O₃), and iron oxide (Fe₂O₃), and depending on the ratios of these components, a hardening speed during hardening of the dental composition or a hardened body strength varies.

As the Portland cement, a conventional Portland cement powder can be used without any restriction, but Portland cement particularly having such composition as below is more preferable because when a kneaded material is hardened, it can be hardened in a stable state and a moderate strength can be obtained, during use under the condition of a large amount of moisture, such as root canal filling or pulp capping. Other components, such as magnesium oxide and sodium carbonate, may be contained.
Calcium oxide (CaO): 55 mass % or more and 85 mass % or less
Silicon dioxide (SiO₂): 10 mass % or more and 40 mass % or less
Aluminum oxide (Al₂O₃): 0 mass % or more and 15 mass % or less
Iron oxide (Fe₂O₃): 0 mass % or more and 10 mass % or less
Gypsum: 0 mass % or more and 20 mass % or less

The type of the Portland cement is not particularly limited, but for example, Portland cements of 6 types of ordinary, rapid hardening, extra rapid hardening, moderate heat, low heat, and sulfate resistant, which are stipulated by JIS R 5210, and their respective low-alkali type Portland cements can be used.

As the Portland cement, I type Portland cement stipulated by ASTM C150, in other words, ordinary Portland cement stipulated by the above JIS, is preferably used from the viewpoint that it has tight sealing properties, exhibits good biocompatibility and has high pH.

An average particle diameter of the cement component is preferably 1 to 100,000 nm, more preferably 100 to 10,000 nm, still more preferably 200 to 3,000 nm, still much more preferably 300 to 1,000 nm, and particularly preferably 400 to 800 nm, though it is not particularly limited. When the average particle diameter of the cement component is in the above range, a balance between a setting time and ease of penetration into dentinal tubule tends to be improved.

The average particle diameter of the cement component refers to a value measured through scanning electron microscopic observation under the general conditions.

It is preferable that the cement component should contain substantially no tetracalcium aluminoferrite. If the cement component contains tetracalcium aluminoferrite, the color of cement turns gray, and therefore, aesthetic quality tends to be deteriorated. Here, the expression “contain substantially no” means that the content is not particularly limited if the content is not more than a content that does not exert influence on the aesthetic quality.

A content of the cement component based on the total solids of the dental composition of the present embodiment is preferably 50 to 95 mass %, more preferably 60 to 90 mass %, and still more preferably 70 to 80 mass %, though it is not particularly limited. When the content of the cement component is 50 mass % or more, the strength of the hardened material tends to be increased, and when it is 95 mass % or less, making a paste tends to be facilitated.

Here, the “solids of the dental composition” refer to components excluding a solvent in the dental composition unless otherwise noted.

(Titanium Oxide, Pure Titanium, Sodium Titanate, and Strontium Titanate)

The dental composition of the present embodiment contains, in addition to the aforementioned cement component, at least one selected from the group consisting of titanium oxide, pure titanium, sodium titanate, and strontium titanate. These components have properties that they have good biocompatibility and simultaneously have excellent X-ray contrast properties and bleaching activity. The present inventors have found for the first time that by incorporating at least one selected from the group consisting of titanium oxide, pure titanium, sodium titanate, and strontium titanate in the dental composition, a dental composition having all of excellent contrast properties, discoloration resistance and compression strength can be obtained.

An average particle diameter of the titanium oxide is preferably 1 to 10,000 nm, more preferably 1 to 1,000 nm, still more preferably 3 to 100 nm, still much more preferably 5 to 50 nm, even much more preferably 6 to 30 nm, and particularly preferably 10 to 30 nm, though it is not particularly limited. When the average particle diameter of the titanium oxide is in the above range, a balance between contrast properties and handling properties tends to be improved.

The pure titanium is not particularly limited, and for example, pure titanium classified as Grade 1 to Grade 4 in JIS standard can be used. A content of titanium in the pure titanium is preferably 98.0 mass % or more, more preferably 99.0 mass % or more, still more preferably 99.5 mass % or more, still much more preferably 99.8 mass % or more, and particularly preferably 99.9 mass % or more. In the pure titanium, an element other than titanium may be partially contained, and examples of such elements include Cl, Mg, Fe, Mn, Si, N, C, H and O.

As the pure titanium, pure titanium in any shape of irregular and spherical shapes can be used, but from the viewpoint that the effect of the present invention becomes more prominent, a spherical shape is preferable. An average particle diameter of the pure titanium is not particularly limited, and the lower limit may be 1 nm or more, or may be 10 nm or more, 30 nm or more, 100 nm or more, 500 nm or more, or 1 μm or more. The upper limit of the average particle diameter is not particularly limited, and it may be 300 μm or less, or may be 200 μm or less, 100 μm or less, 50 μm or less, 30 μm or less, or 10 μm or less.

As the pure titanium, a commercial product may be used, and examples of the commercial products include titanium powders manufactured by TOHO TECHNICAL SERVICE CO., LTD. (TC-150, TC-450, TC-459, TC-201, TC-15S, TC-45S, etc.).

Average particle diameters of the sodium titanate and the strontium titanate were each preferably 1 to 10,000 nm, more preferably 1 to 1,000 nm, still more preferably 3 to 100 nm, still much more preferably 5 to 50 nm, and particularly preferably 6 to 30 nm, though they are not particularly limited. When the average particle diameters of the sodium titanate and the strontium titanate were each in the above range, a balance between contrast properties and handling properties tends to be improved.

The average particle diameters of the titanium oxide, the pure titanium, the sodium titanate and the strontium titanate each refer to a value measured through scanning electron microscopic observation under the general conditions.

A content of the at least one selected from the group consisting of titanium oxide, pure titanium, sodium titanate and strontium titanate based on the total solids of the dental composition is preferably 5 to 30 mass %, more preferably 10 to 25 mass %, and still more preferably 15 to 22 mass %. When the content of the at least one selected from the group consisting of titanium oxide, pure titanium, sodium titanate and strontium titanate is in the above range, a balance between biocompatibility and contrast properties tends to be improved.

(Calcium Sulfate-Containing Inorganic Substance)

The dental composition of the present embodiment may further contain at least one calcium sulfate-containing inorganic substance selected from the group consisting of calcium sulfate dihydrate, calcium sulfate hemihydrate, anhydrous calcium sulfate, and gypsum, separately from one contained in the cement component. When the dental composition contains the aforementioned calcium sulfate-containing inorganic substance, chemical reaction can be promoted, and therefore, the setting time tends to be able to be shortened.

A content of the calcium sulfate-containing inorganic substance (excluding one contained in the cement component) based on the total solids of the dental composition is preferably 0 to 10 mass %, more preferably 2 to 8 mass %, and still more preferably 3 to 7 mass %, though it is not particularly limited. When the content of the calcium sulfate-containing inorganic substance is in the above range, not only can hardening be accelerated, but also more sufficient hardness tends to be obtained.

The dental composition of the present embodiment may further contain other components, such as a polymerization initiator, a filler, an antibacterial agent, a coloring agent, a stabilizer and a solvent, as long as the effect of the present invention is not hindered.

It is preferable that the dental composition of the present embodiment should contain substantially no bismuth oxide. If the dental composition contains bismuth oxide, the color of the composition easily turns black when the composition is exposed to light, and the aesthetic quality tends to be deteriorated. Here, the expression “contain substantially no” means that the content is not particularly limited if the content is not more than a content that does not exert influence on the aesthetic quality.

The dental composition of the present embodiment may contain substantially no zirconium oxide. According to the present embodiment, the dental composition contains at least one selected from the group consisting of titanium oxide, pure titanium, sodium titanate and strontium titanate, and therefore, not only are the contrast properties sufficient but also a balance among contrast properties, discoloration resistance and compression strength becomes excellent, so that there is no need to use zirconium oxide. Here, the expression “contain substantially no” means that the content is not particularly limited if the content is not more than a content that does not exert influence on the balance among contrast properties, discoloration resistance and compression strength.

(Polymerization Initiator)

As the polymerization initiator, a general polymerization initiator that is used for dental applications can be used, and in particular, a photopolymerization initiator is preferable. Specific examples of the polymerization initiators include an organic peroxide, an inorganic peroxide, a diazo-based compound, and an organoboron compound.

Examples of the organic peroxides include alkyl peroxides, such as isobutyl peroxide and decanoyl peroxide; peroxycarboxylic anhydrides, such as acetyl peroxide; aromatic peroxycarboxylic anhydrides, such as benzoyl peroxide; peroxyanhydrides of polycarboxylic acids, such as succinic acid peroxide; peroxydicarbonates, such as diisopropyl peroxydicarbonate, di-2-ethylhexyl peroxydicarbonate, and diallyl peroxydicarbonate; peroxyesters, such as tert-butyl peroxyisobutyrate, tert-butyl peroxyneodecanoate, and cumene peroxyneodecanoate; and peroxyanhydrides of carboxylic acids and sulfonic acid, such as acetylcyclohexylsulfonyl peroxide.

Examples of the inorganic peroxides include ammonium persulfate, potassium persulfate, and potassium perphosphate.

Examples of the diazo-based compounds include 2,2′-azobisisobutyronitrile, 4,4′-azobis(4-cyanovaleric acid), 2,2′-azobis(4-methoxy-2,4-dimethoxyvaleronitrile), and 2,2′-azobis(2-cyclopropylpropionitrile).

Examples of the photopolymerization initiators include camphor quinone-based compounds, such as an α-diketone compound and camphor quinone; naphthyl-based compounds, such as α-naphthyl; benzyl-based compounds, such as benzyl and p,p′-dimethoxybenzyl: b-diketone compounds, such as pentadione; quinone-based compounds, such as 1,4-phenanthrenequinone and naphthoquinone; benzoylphosphine oxide-based compounds, such as diphenyltrimethylbenzoylphosphine oxide; and tertiary amine compounds.

The polymerization initiators may be used singly or may be used in combination of two or more. A content of the polymerization initiator is preferably 0.01 to 40 mass %, more preferably 0.05 to 35 mass %, and still more preferably 0.1 to 30 mass %, based on the total solids of the dental composition, though it is not particularly limited. When the content is in the above numerical range, the polymerization rate is in an appropriate range, the therapeutic treatment time is sufficiently secured, and insufficient hardening due to shortage of the polymerization initiator tends to hardly occur.

(Filler)

Examples of the fillers include an inorganic filler, an organic filler, and a composite filler of these. Examples of the fillers include zinc oxide, crushed quartz, wet silica, dry silica, carbon black, diatomaceous earth, a glass fiber, alumina, magnesia, magnesium carbonate, titanium, zinc, zirconium, zirconium oxide, zirconium dioxide, strontium, tin, barium, tungsten, bismuth, a pulverized polymer, and a powder polymer.

As the filler, a filler containing a substance that is opaque to X-rays and has X-ray contrast properties may be used, and examples of such fillers include lithium, bismuth, barium, strontium, zirconium, and oxides of these (e.g., zirconium oxide). The inorganic filler may be subjected to surface treatment, such as silane treatment or polymer coating.

Among the above, at least one selected from the group consisting of silica and zirconium oxide is preferable as the filler from the viewpoint that the dental composition has more excellent mechanical strength and adhesion to an adherend, and silica is more preferable as the filler from the viewpoint that the dental composition has excellent flexural strength.

The fillers may be used singly or may be used in combination of two or more. A content of the filler is preferably 0.1 mass % to 30 mass %, more preferably 1 mass % to 20 mass %, and still more preferably 1 mass % to 10 mass %, based on the total solids of the dental composition. When the content is in the above numerical range, the strength of the hardened material tends to become sufficient.

(Antibacterial Agent)

The dental composition of the present embodiment may contain an antibacterial agent for the purpose of preventing a secondary infection of dental pulp and root canal. As the antibacterial agent, an inorganic antibacterial agent or an organic antibacterial agent can be used.

Examples of the inorganic antibacterial agents include synthetic zeolite supporting thereon an inorganic ion such as a silver ion, a copper ion or a zinc ion, inorganic ion-supported calcium phosphate, inorganic ion-supported vanadium phosphate, inorganic ion-supported calcium silicate, inorganic ion-supported silica gel, inorganic ion-supported zirconium phosphate, inorganic ion-supported amorphous, inorganic ion-supported titanium oxide, and an inorganic ion-supported oxide photocatalyst.

Examples of the organic antibacterial agents include a phenol ether derivative, a sulfone derivative, an imidazole derivative, Eugenol, and polymerizable monomers having a cationic group, such as (meth)acryloyloxy hexadecylpyridinium bromide, (meth)acryloyloxy hexadecylpyridinium chloride, and (meth)acryloyloxy decylammonium chloride.

The antibacterial agents may be used singly or may be used in combination of two or more. A content of the antibacterial agent is preferably 0.1 mass % to 10 mass % based on the total solids of the dental composition. When the content is in the above numerical range, antibacterial properties commensurate with the amount added is easily obtained.

(Coloring Agent)

The dental composition of the present embodiment may contain a coloring agent. Examples of the coloring agents include Phloxine BK, Acid Red, Fast Acid Magenta, Phloxine B, Fast Green FCF, Rhodamine B, basic fuchsine, acid fuchsine, eosin, erythrosine, safranin, rose bengal, bemer, gentian violet, copper chlorophyll soda, laccaic acid, fluorescein sodium, cochineal, shisonin, talc, and titanium white. The coloring agents may be used singly or may be used in combination of two or more.

(Stabilizer)

The dental composition of the present embodiment may contain a stabilizer. Examples of the stabilizers include hydroquinone compounds, such as hydroquinone, dibutyl hydroquinone and hydroquinone monomethyl ether, and phenols, such as 2,6-di-tert-butylphenol and 2,6-di-tert-butyl-p-cresol. The stabilizers may be used singly or may be used in combination of two or more.

(Solvent)

The dental composition of the present embodiment may contain a solvent for purposes such as improvement in solubility of the constituents, viscosity preparation, and improvement in affinity for an adherend. Examples of the solvents include water and ethanol. A content of the solvent is preferably 1 mass % to 50 mass % based on the whole dental composition.

[Method for Producing Dental Composition]

A method for producing the dental composition of the present embodiment is not particularly restricted, and a known method can be adopted. For example, a method including mixing the aforementioned components to produce the dental composition, and a method including adding the aforementioned various components to a prescribed solvent to dissolve them, and thereafter removing the solvent as needed to produce the dental composition can be mentioned.

[Method for Using Dental Composition]

Next, a method for using the dental composition of the present embodiment will be described.

The dental composition of the present embodiment can be used in the same manner as for the conventional dental composition (root canal treatment material). Hereinafter, a general procedure of root canal treatment in the case where the dental composition of the present embodiment is used as a pulp capping material or a root canal filling material will be described, but the method for using the dental composition of the present embodiment is not limited thereby.

In the case where the dental composition is used as a pulp capping material, first, a rubber sheet for moisture exclusion called a rubber dam is placed in the oral cavity of a patient, the rubber dam at the affected tooth portion is drilled, and only the affected tooth is exposed from the rubber dam using a clamp. Thereafter, the carious dentin of the affected tooth is removed, then the cavity is subjected to irrigation and sterilization using a sodium hypochlorite solution and a hydrogen peroxide solution and further subjected to irrigation using a sterile physiological saline solution, and the cavity is dried with a sterile swab. At this time, the affected tooth is close to the dental pulp, or the dental pulp is exposed, so that drying of the cavity is sometimes insufficient due to an exudate fluid or the like. Thereafter, the dental composition of the present embodiment is applied to the exposed pulp surface using an instrument called an applicator. The dental composition is irradiated with light and thereby set, and thereafter, filling, crown restoration or the like using glass ionomer cement or the like is carried out as needed to finish the treatment.

Also, in the case where the dental composition is used as a root canal filling material, a rubber dam is placed in the oral cavity of a patient similarly to the above, and the root canal in the affected tooth, which has been exposed from the rubber dam, is subjected to irrigation with a sodium hypochlorite solution and a hydrogen peroxide solution. Thereafter, the root canal is dried with a broach cotton plug or a paper point. In this case also, drying sometimes becomes insufficient for the same reason as above. Moreover, the shape of the root canal varies depending on the patient, the site, or the individual tooth, and therefore, if the root canal is curved, sufficient drying sometimes becomes much more difficult. After drying of the root canal, the dental composition is applied to a needle-like member made of rubber, which is called a gutta-percha point (main point), and the member is inserted to the prescribed position of the root canal. Subsequently, another gutta-percha point (accessary point) is inserted into a gap as needed, thereafter, a pressure is applied to the main point laterally with an instrument called a spreader, and into a gap formed, an accessory point is further inserted, and these operations are continued until the root canal can be filled without any space. Thereafter, crown restoration or the like is carried out to finish the treatment. The dental composition of the present embodiment can also be used in, in addition to the above methods, a method in which the root canal is directly filled with the dental composition by an applicator or the like without using a gutta-percha point.

In the root canal treatment, as described above, it is sometimes difficult to completely remove moisture from the dental pulp or the root canal that is an adherend, but by using the dental composition of the present embodiment, the root canal treatment material firmly adheres to the dental pulp or the root canal dentin even in a wet environment to form a stable hardened material, so that the dental pulp or the root canal can be sealed, and causing of a secondary infection in the dental pulp or the root canal can be prevented after the root canal treatment.

### EXAMPLES

Hereinafter, the present invention will be specifically described with examples. However, the present invention is not limited thereto. Unless otherwise noted, “part(s)” and “%” are each on a mass basis.

### Example 1

Portland cement (manufactured by Lehigh White Cement Company, trade name: White Portland Cement) was pulverized into nano size (average particle diameter: 300 to 1,000 nm). With this, titanium oxide (manufactured by ISHIHARA SANGYO KAISHA, LTD., trade name: Ultrafine titanium oxide TTO-51(A), average particle diameter: 10 nm to 30 nm) was mixed in such a manner that the proportion thereof became 15 mass % based on the whole composition, thereby obtaining a dental composition.

### Comparative Example 1

A dental composition containing Portland cement and bismuth oxide (manufactured by Dentsply Sirona Inc., trade name: ProRoot MTA Gray) was used.

(Measurement of Degree of Discoloration)

Ten extracted teeth were each subjected to root canal preparation up to #35/0.06 using Vortex Blue. After root canal irrigation with 17% EDTA and 6% NaOCl, distilled water and a powder of the dental composition obtained in Example 1 were mixed in a ratio of 1:3 and kneaded, and using this, root canal filling was carried out. Subsequently, the teeth were stored in a container containing a physiological saline solution, in a sealed state.

Values (%) for the degree of discoloration of images obtained by photographing (Sony α6500) the teeth under a Zeiss PROergo microscope before root canal filling, and 1 month, 3 months, 6 months and 12 months after root canal filling were measured using Grayscale of Photoshop and compared. Through two-way analysis of variance, statistical processing was carried out. As a result, before root canal filling, an average value of the degrees of discoloration was 32%, 1 month after root canal filling, it was 33%, 3 months after that, it was 35%, 6 months after that, it was 35%, and 12 months after that, it was 36%, and a statistically significant difference was not observed (p<0.05). That is to say, discoloration was not observed in the dental composition of Example 1.

(Measurement of Degree of Contrast)

Ten extracted teeth were each subjected to root canal preparation up to #35/0.06 using Vortex Blue. After root canal irrigation with 17% EDTA and 6% NaOCl, distilled water and a powder of the dental composition obtained in Example 1 or Comparative Example 1 were mixed in a ratio of 1:3 and kneaded. Using a substance obtained by using the dental composition of Example 1, root canal filling of 5 extracted teeth was carried out, and using a substance obtained by using the dental composition of Comparative Example 1, root canal filling of the remaining 5 extracted teeth was carried out. X-ray photographs were taken by Trophy Windows.

The X-ray photographs taken were recorded in JPEG, and values (%) for the degree of contrast were measured using Grayscale in Photoshop and compared. Through two-way analysis of variance, statistical processing was carried out. As a result, an average value of the degrees of contrast of Example 1 was 27%, and that of Comparative Example 1 was 21%, and a statistically significant difference was not observed (p<0.05). That is to say, it has been understood that the contrast properties of the dental composition of Example 1 were almost equal to the contrast properties of the dental composition of Comparative Example 1.

(Measurement of Compression Strength)

The dental compositions obtained in Example 1 and Comparative Example 1 in each amount of 1 g were each kneaded with 0.34 g of distilled water, and with each of these, 10 PTFE tubes (height: 6±0.1 mm, inner diameter: 4±0.1 mm) were filled. They were stored at 37° C. and a humidity of 100%. After 7 days, hardening was confirmed.

In the measurement of compression strength, a pressure was applied to the long axis of the hardened cement at a rate of 1 mm/min using a general compression force measuring device (Lloyd Instruments Ltd., Fareham, UK), and a strength given when the cement was fractured was recorded as a compression strength (MPa). The compression strength was calculated from 4P/πr2, and differences in compression strength were subjected to statistical processing (p<0.05). Through two-way analysis of variance, statistical processing was carried out. As a result, an average value of the compression strengths of Example 1 was 97.38±23.91 MPa, and that of Comparative Example 1 was 68.88±28.34 MPa, and when the compression strength of the dental composition of Example 1 was compared with the compression strength of the dental composition of Comparative Example 1, a statistically significant difference was observed (p<0.05). That is to say, the compression strength of the dental composition of Example 1 was significantly higher than the compression strength of the dental composition of Comparative Example 1.

### Example 2

A dental composition was obtained in the same manner as in Example 1, except that the titanium oxide was mixed in such a manner that the proportion thereof became 20 mass % based on the whole composition.

The degree of discoloration was measured in the same manner as above, and as a result, before root canal filling, an average value of the degrees of discoloration was 33%, 1 month after root canal filling, it was 35%, 3 months after that, it was 37%, 6 months after that, it was 35%, and 12 months after that, it was 36%, and a statistically significant difference was not observed (p<0.05). That is to say, discoloration was not observed in the dental composition of Example 2.

The degree of contrast was measured in the same manner as above, and as a result, an average value of the degrees of contrast of Example 2 was 26%, and that of Comparative Example 1 was 21%, and a statistically significant difference was not observed (p<0.05). That is to say, it has been understood that the contrast properties of the dental composition of Example 2 were almost equal to the contrast properties of the dental composition of Comparative Example 1.

The compression strength was measured in the same manner as above, and as a result, an average value of the compression strengths of Example 2 was 101.12±22.54 MPa, and that of Comparative Example 1 was 68.88±28.34 MPa, and when the compression strength of the dental composition of Example 2 was compared with the compression strength of the dental composition of Comparative Example 1, a statistically significant difference was observed (p<0.05). That is to say, the compression strength of the dental composition of Example 2 was significantly higher than the compression strength of the dental composition of Comparative Example 1.

### Example 3

A dental composition was obtained in the same manner as in Example 1, except that the titanium oxide was mixed in such a manner that the proportion thereof became 25 mass % based on the whole composition.

The degree of discoloration was measured in the same manner as above, and as a result, before root canal filling, an average value of the degrees of discoloration was 31%, 1 month after root canal filling, it was 27%, 3 months after that, it was 28%, 6 months after that, it was 28%, and 12 months after that, it was 30%, and a statistically significant difference was not observed (p<0.05). That is to say, discoloration was not observed in the dental composition of Example 2.

The degree of contrast was measured in the same manner as above, and as a result, an average value of the degrees of contrast of Example 3 was 23%, and that of Comparative Example 1 was 21%, and a statistically significant difference was not observed (p<0.05). That is to say, it has been understood that the contrast properties of the dental composition of Example 3 were almost equal to the contrast properties of the dental composition of Comparative Example 1.

The compression strength was measured in the same manner as above, and as a result, an average value of the compression strengths of Example 3 was 121.12±20.14 MPa, and that of Comparative Example 1 was 68.88±28.34 MPa, and when the compression strength of the dental composition of Example 3 was compared with the compression strength of the dental composition of Comparative Example 1, a statistically significant difference was observed (p<0.05). That is to say, the compression strength of the dental composition of Example 3 was significantly higher than the compression strength of the dental composition of Comparative Example 1.

1. A dental composition comprising
a cement component, and
at least one selected from the group consisting of titanium oxide, pure titanium, sodium titanate, and strontium titanate.

2. The dental composition according to claim 1, wherein the cement component is Portland cement.

3. The dental composition according to claim 1, wherein an average particle diameter of the cement component is 1 to 100,000 nm.

4. The dental composition according to claim 1, wherein the cement component contains substantially no tetracalcium aluminoferrite.

5. The dental composition according to claim 1, wherein a content of the cement component is 50 to 95 mass %, and a content of the at least one selected from the group consisting of titanium oxide, pure titanium, sodium titanate, and strontium titanate is 5 to 30 mass %, based on the total solids of the dental composition.

6. The dental composition according to claim 1, wherein a content of the at least one selected from the group consisting of titanium oxide, pure titanium, sodium titanate, and strontium titanate is 10 to 25 mass %.

7. The dental composition according to claim 1, wherein an average particle diameter of the titanium oxide is 3 to 100 nm.

8. The dental composition according to claim 1, wherein a content of titanium in the pure titanium is 99.9 mass % or more.

9. The dental composition according to claim 1, wherein an average particle diameter of the pure titanium is 1 nm to 300 μm.

10. The dental composition according to claim 1, further comprising at least one calcium sulfate-containing inorganic substance selected from the group consisting of calcium sulfate dihydrate, calcium sulfate hemihydrate, anhydrous calcium sulfate, and gypsum.

11. The dental composition according to claim 10, wherein a content of the calcium sulfate-containing inorganic substance based on the total solids of the dental composition is 0 to 10 mass %.

12. The dental composition according to claim 10, wherein a content of the calcium sulfate-containing inorganic substance based on the total solids of the dental composition is 2 to 8 mass %.

13. The dental composition according to claim 1, used as a root canal treatment material.
